(19) United States
(12) Patent Application Publication
(10) Pub. No.: US 2019/0358094 A1
(43) Pub. Date: Nov. 28, 2019

(54) DISPOSABLE ABSORBENT ARTICLE AND METHOD OF PRODUCING THE SAME

(71) Applicant: SJ CIRCLE SDN. BHD., Kulim (MY)

(72) Inventor: Sew Jin YEOH, Butterworth (MY)

(21) Appl. No.: 16/419,460

(22) Filed: May 22, 2019

(30) Foreign Application Priority Data
May 25, 2018 (MY) PI2018702040

(51) Int. Cl. A61F 13/511; A61F 13/534; A61F 13/532

(52) U.S. Cl. CPC A61F 13/511; A61F 2013/530007; A61F 13/5323; A61F 13/534

(57) ABSTRACT

The present invention relates generally to a system of producing a disposable absorbent article comprising a top sheet, a back sheet, an absorbent core positioned between said top sheet and said back sheet wherein said absorbent core having a core wrap that fully envelopes said absorbent core; said absorbent core comprises a mixture of fluff pulp and superabsorbent polymer (SAP) wherein said absorbent core having composition of SAP higher than composition of fluff pulp, and said SAP is having an Absorbency Under Load (AUL) above 17 g/g at 0.7 psi.

### 1. TECHNICAL FIELD OF THE INVENTION

The present invention relates generally to a system of producing a disposable absorbent article comprising a top sheet, a back sheet, an absorbent core positioned between said top sheet and said back sheet wherein said absorbent core having a core wrap that fully envelopes said absorbent core; said absorbent core comprises a mixture of fluff pulp and superabsorbent polymer (SAP) wherein said absorbent core having composition of SAP higher than composition of fluff pulp, and said SAP is having an Absorbency Under Load (AUL) above 17 g/g at 0.7 psi.

### 2. BACKGROUND OF THE INVENTION

Absorbent articles, such as diapers, adult incontinence products, and training pants are designed to absorb and retain body fluids or liquids such as urine, feces or the like. Nevertheless, absorbent pad which is made of polyurethane foam having a structure that is relatively bulky and might cause a bigger size of absorbent articles.

Tanzer, U.S. Pat. No. 5,411,497 A disclosed an absorbent article comprises a first, liquid-permeable carrier layer and at least a second carrier layer. A water sensitive attaching mechanism secures together the carrier layers to provide substantially attached zones and substantially unattached zones thereof. The substantially unattached zones provide a plurality of pocket regions, and a high absorbency material is located within the pocket regions to provide an absorbent laminate. However, due to the construction of the pockets, and specifically due to the selection of the water-sensitive attachment means, these pockets are not maintained when the article is fully or partially loaded with liquids. Hence, this absorbent article does not provide a satisfactory immobilization of the absorbent material in the fully or partially urine loaded state.

Holmes et. al., US 20020115971 A1, discloses a thin, high capacity, multi-layer absorbent material. An upper layer displays intake property while a lower layer displays distribution property. The upper and lower layers are both high density combinations of fluff pulp and superabsorbent material, with the lower layer having a higher density than the upper layer. The upper layer is suitably drum-formed, the lower layer is suitably air-laid, and additional layers can be either inserted between the upper and lower layers or added on top of the upper layer. The absorbent material can be used to provide discreet absorbent protection in absorbent articles, such as diapers, training pants, feminine hygiene products, incontinence products, and swim wear. However, said prior art does not disclosed thin absorbent core with high absorbency.

Hence, it would be advantageous to alleviate the shortcomings by having a system and method of producing thin absorbent core with high level of superabsorbent polymer (SAP) and Absorbency Under Load (AUL).

### 3. SUMMARY OF THE INVENTION

Accordingly, it is the primary aim of the present invention to provide a disposable absorbent article with high level of superabsorbent polymer (SAP).

It is yet another object of the present invention to provide a disposable absorbent article having thin absorbent core with high absorbency.

It is yet another object of the present invention is to provide a disposable absorbent article which is environmental friendly.

It is yet another object of the present invention is to provide a disposable absorbent article which is user friendly.

Other further objects of the invention will become apparent with an understanding of the following detailed description of the invention or upon employment of the invention in practice.

According to the preferred embodiment of the present invention the following is provided:

A disposable absorbent article comprising:
a top sheet;
a back sheet; and
an absorbent core positioned between said top sheet and said back sheet wherein said absorbent core having a core wrap that fully envelopes said absorbent core;

characterized in that;
said absorbent core comprises of a mixture of fluff pulp and superabsorbent polymer (SAP) wherein said absorbent core having composition of SAP higher than composition of fluff pulp; and
said SAP having an Absorbency Under Load (AUL) above 17 g/g at 0.7 psi.
   In another embodiment of the invention there is provided:

A method (3) of producing absorbent core comprising the steps of:
(i) mixing at least two superabsorbent polymer (SAP) within a chamber (301);
(ii) mixing said mixture of SAP particles with fluff pulp wherein said mixture of SAP particles and fluff pulp having composition of SAP particles higher than composition of fluff pulp and said SAP particles having an Absorbency Under Load (AUL) above 17 g/g at 0.7 psi (303);
(iii) applying at least one adhesive material to at least one core wrap (305); and
(iv) fully enveloping said mixture of SAP particles and fluff pulp with said core wrap (307).

### 4. BRIEF DESCRIPTION OF THE DRAWINGS

Other aspect of the present invention and their advantages will be discerned after studying the Detailed Description in conjunction with the accompanying drawings in which:

FIG. 1-A shows an exemplary view of a disposable absorbent article.

FIG. 1-B shows an exemplary view of an absorbent core.

FIG. 2 shows an exemplary diagram of a system of producing absorbent core.

FIG. 3 shows an exemplary method flow diagram of the present invention.

### DETAILED DESCRIPTION OF THE DRAWINGS

In the following detailed description, numerous specific details are set forth in order to provide a thorough understanding of the invention. However, it will be understood by those with ordinary skill in the art that the invention may be practiced without these specific details. In other instances, well known methods, procedures and/or components have not been described in detail so as not to obscure the invention.

The invention will be more clearly understood from the following description of the embodiments thereof, given by way of example only with reference to the accompanying drawings, which are not drawn to scale.

As used in this disclosure and the appended claims herein, the singular forms “a”, “an”, and “the” include plural referents unless the context clearly dictates or denotes otherwise.

Throughout the disclosure and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment, “such as” is not used in a restrictive sense, but for explanatory purposes.

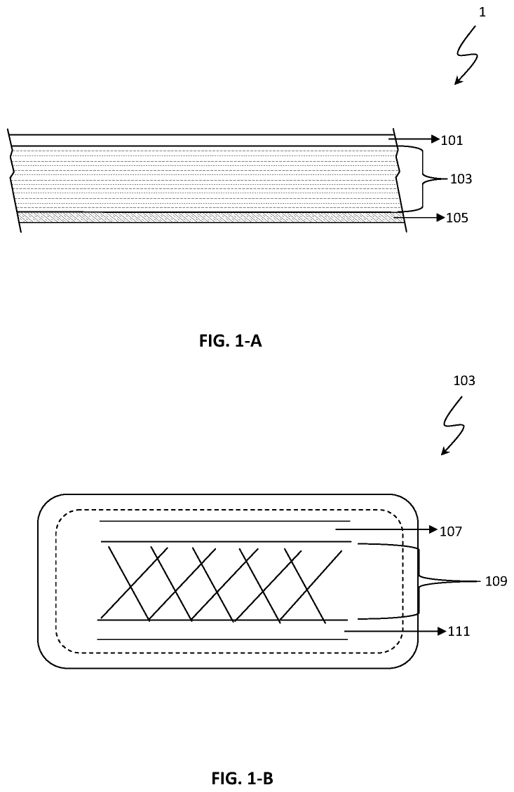

Referring to FIG. 1-A, there is shown an exemplary view of a disposable absorbent article wherein said disposable absorbent article (1) comprising of a top sheet (101); a back sheet (105); and an absorbent core (103) positioned between said top sheet (101) and said back sheet (105) wherein said absorbent core (103) having a core wrap that fully envelopes said absorbent core (103); said absorbent core (103) comprises of a mixture of fluff pulp and superabsorbent polymer (SAP) wherein said absorbent core (103) having composition of SAP higher than composition of fluff pulp; and said SAP particles having an Absorbency Under Load (AUL) above 17 g/g at 0.7 psi. In addition, said mixture of fluff pulp and SAP is adhered by core integrity glue. Said SAP particles further comprises of different shapes of neighbouring particles.

Referring to FIG. 1-B, there is shown an exemplary view of an absorbent core wherein said absorbent core (103) comprises of a first fluff zone (107); a mixed zone (109); and a second fluff zone (111); wherein said first fluff zone (107) and said second fluff zone (111) comprise of 0% to 10% by weight of the SAP and remaining being the fluff pulp; whereas said mixed zone (109) comprises of 10% to 90% by weight of the SAP and remaining being the fluff pulp.

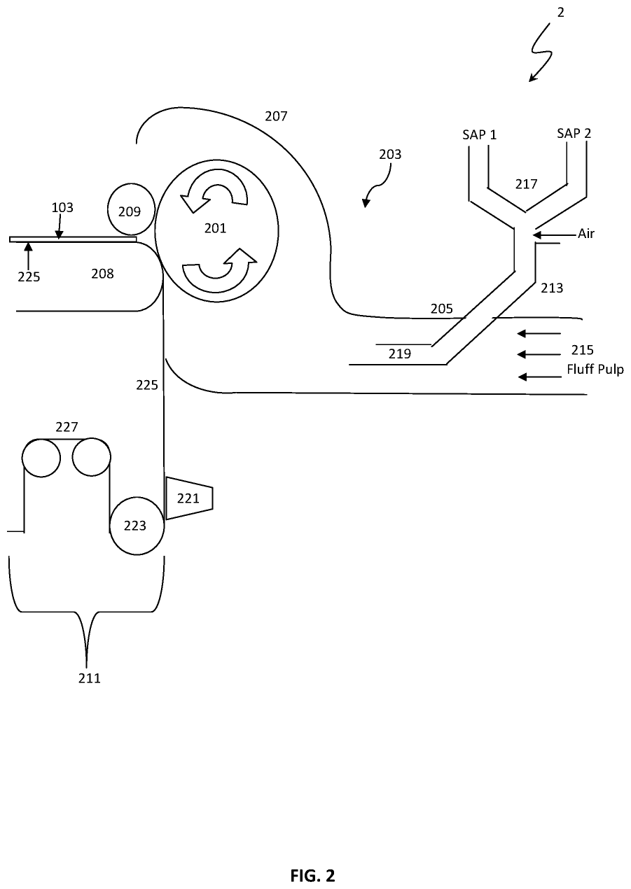

Referring to FIG. 2, there is shown an exemplary diagram of a system of producing absorbent core. Said system of producing absorbent core comprising of at least one rotary suction mechanism (201) having a channel (203) wherein said channel (203) partially wrapping said rotary suction mechanism (201); said channel (203) comprises of a linear portion (205) and a concave portion (207) wherein said linear portion (205) channeling fluff pulp and said concave portion (207) channeling mixture of fluff pulp and superabsorbent polymer (SAP) particles; at least one rotatable roller (209) positioned near the circumference of said rotary suction mechanism (201); said system (2) further comprises of multiple input stations wherein said input stations include core wrap station (211), superabsorbent polymer (SAP) station (213), and fluff pulp station (215); said SAP station (213) comprises of at least one chamber (217) with multiple inlets (SAP 1, SAP 2) located at the top of said chamber (217) wherein said chamber (217) connects with an input from said fluff pulp station (215) at said linear portion (205); said chamber (217) further comprises of an outlet (219) wherein said outlet (219) meets direction of said fluff pulp at said linear portion (205) before directing said SAP particles and fluff pulp at an angle away from direction of said fluff pulp towards the upper circumference of said rotary suction mechanism (201) along the concave portion (207); and said outlet (219) positioned substantially centre of axis of said linear portion (205) of said channel (203). Said outlet (219) having an upper wall and a lower wall wherein said upper wall is shorter than said lower wall.

In addition, said system (2) further comprises of a core integrity glue station (221) positioned substantially nearby said core wrap station (211) wherein said core integrity glue station (221) providing glue to the surface of a wrap facing mixture of fluff pulp and SAP particles. Said core wrap station (211) comprises of at least one rotatable roller (223). A wrap (225) is moving continuously between said rotatable roller (223) of said core wrap station (211) and said rotatable roller (209) of said rotary suction mechanism (201). Said core wrap station (211) further comprises of a web guide (227) for alignment of said wrap (225). Said rotatable roller (223) of said core wrap station (211) is guiding said wrap (225) toward a conveyor (208) and said rotatable roller (209) of said rotary suction mechanism (201) to enable the wrapping process of said absorbent core (103). Besides, said rotatable roller (209) of said rotary suction mechanism (201) is guiding said absorbent core (103) to a conveyor (208) to enable said absorbent core (103) to be fully enveloped by said wrap (225).

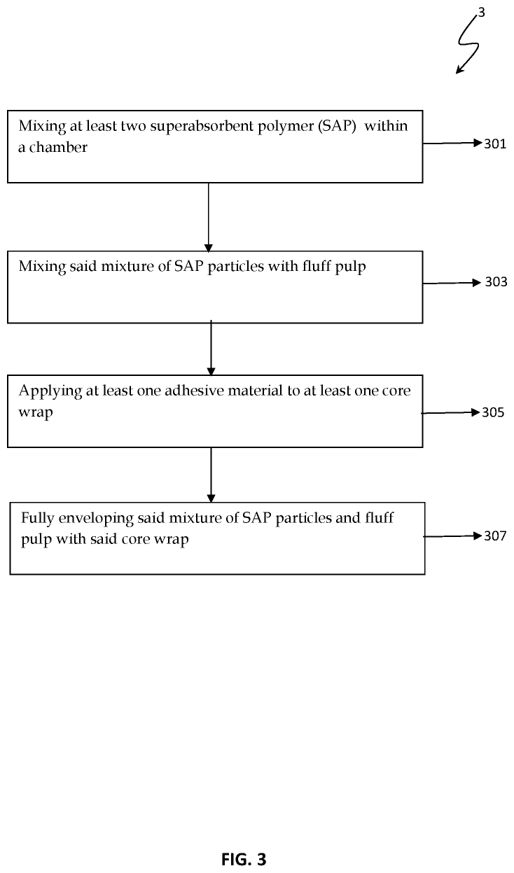

Referring to FIG. 3, there is shown an exemplary method flow diagram of the present invention which comprises of the steps of: mixing at least two superabsorbent polymer (SAP) within a chamber (301), mixing said mixture of SAP particles with fluff pulp wherein said mixture of SAP particles and fluff pulp having composition of SAP particles higher than composition of fluff pulp and said SAP particles having an Absorbency Under Load (AUL) above 17 g/g at 0.7 psi (303), applying at least one adhesive material to at least one core wrap (305), and fully enveloping said mixture of SAP particles and fluff pulp with said core wrap (307). Said superabsorbent polymer (SAP) includes superabsorbent polymer (SAP) with different particles shape.

While the present invention has been shown and described herein in what are considered to be the preferred embodiments thereof, illustrating the results and advantages over the prior art obtained through the present invention, and the invention is not limited to those specific embodiments. Thus, the forms of the invention shown and described herein are to be taken as illustrative only and other embodiments may be selected without departing from the scope of the present invention, as set forth in the claims appended hereto.

1. A disposable absorbent article comprising:
a top sheet;
a back sheet; and
an absorbent core positioned between said top sheet and said back sheet wherein said absorbent core having a core wrap that fully envelopes said absorbent core;
characterized in that;
said absorbent core comprises of a mixture of fluff pulp and superabsorbent polymer (SAP) particles wherein said absorbent core having composition of SAP particles higher than composition of fluff pulp; and
said SAP particles having an Absorbency Under Load (AUL) above 17 g/g at 0.7 psi.

2. The disposable absorbent article as claimed in claim 1 wherein said absorbent core further comprise of:
a first fluff zone;
a mixed zone; and
a second fluff zone;
said first fluff zone and said second fluff zone comprise of 0% to 10% by weight of the SAP particles and remaining being the fluff pulp;
said mixed zone comprises of 10% to 90% by weight of the SAP particles and remaining being the fluff pulp.

3. The disposable absorbent article as claimed in claim 1 wherein said mixture of fluff pulp and SAP particles is adhered by core integrity glue.

4. The disposable absorbent article as claimed in claim 1 wherein said SAP particles further comprises of different shapes of neighbouring particles.

5. A method of producing absorbent core comprising the steps of:
(i) mixing at least two superabsorbent polymer (SAP) within a chamber;
(ii) mixing said mixture of SAP particles with fluff pulp wherein said mixture of SAP particles and fluff pulp having composition of SAP particles higher than composition of fluff pulp and said SAP particles having an Absorbency Under Load (AUL) above 17 g/g at 0.7 psi;
(iii) applying at least one adhesive material to at least one core wrap; and
(iv) fully enveloping said mixture of SAP particles and fluff pulp with said core wrap.

6. The method of producing absorbent core as claimed in claim 5 wherein said superabsorbent polymer (SAP) includes superabsorbent polymer (SAP) with different particles shape.

7. The method of producing absorbent core as claimed in claim 5 wherein said adhesive material includes core integrity glue.
